(19) United States
(12) Patent Application Publication
(10) Pub. No.: US 2018/0104381 A1
(43) Pub. Date: Apr. 19, 2018

(54) CONSISTENT CALCIUM CONTENT BONE ALLOGRAFT SYSTEMS AND METHODS

(71) Applicant: AlloSource, Centennial, CO (US)

(72) Inventors: Matthew James Southard, Denver, CO (US); Kenneth Blood, Littleton, CO (US); Marina Katelyn Bull, Highlands Ranch, CO (US); Adrian Samaniego, Centennial, CO (US); Nathan Ness, Denver, CO (US)

(21) Appl. No.: 15/431,309

(22) Filed: Feb. 13, 2017

Related U.S. Application Data
(60) Provisional application No. 62/408,174, filed on Oct. 14, 2016.

(51) Int. Cl. A61L 27/36; A61L 27/50; A61L 27/38; A61L 27/54

(52) U.S. Cl. CPC A61L 27/3608; A61L 27/50; A61F 2/28; A61L 27/54; A61L 27/3834

(57) ABSTRACT

Embodiments of the present invention provides bone graft compositions, and methods for their use and manufacture. A bone graft composition may include a first amount of demineralized cortical bone that includes non-spherical particles. The composition may further include a second amount of demineralized cancellous bone. The composition may also include a third amount of non-demineralized cortical bone. The demineralized cortical bone, the demineralized cancellous bone, and the non-demineralized cortical bone may be obtained from the same cadaveric donor.

### CROSS-REFERENCES TO RELATED APPLICATIONS

This application claims the benefit of U.S. Provisional Patent Application No. 62/408,174 filed Oct. 14, 2016. This application is also related to Provisional Patent Application No. 61/774,036 filed Mar. 7, 2013; U.S. Pat. No. 9,289,452 filed Mar. 7, 2014; and U.S. patent application Ser. No. 14/996,469 filed Jan. 15, 2016, the entire contents of all are incorporated herein by reference for all purposes.

### BACKGROUND

Embodiments of the present invention are directed in general to the field of medical grafts, and in particular to bone graft compositions, and methods of their use and manufacture.

Medical grafting procedures often involve the implantation of autogenous, allograft, or synthetic grafts into a patient to treat a particular condition or disease. The use of musculoskeletal allograft tissue in reconstructive orthopedic procedures and other medical procedures has markedly increased in recent years, and millions of musculoskeletal allografts have been safely transplanted. A common allograft is bone. Typically, bone grafts are reabsorbed and replaced with the patient's natural bone upon healing. Bone grafts can be used in a variety of indications, including neurosurgical and orthopedic spine procedures for example. In some instances, bone grafts can be used to fuse joints or to repair broken bones.

Allograft and autogenous bone are both derived from humans; the difference is that allograft is harvested from an individual (e.g. donor) other than the one (e.g. patient) receiving the graft. Allograft bone is often taken from cadavers that have donated their bone so that it can be used for living people who are in need of it, for example, patients whose bones have degenerated from cancer. Such tissues represent a gift from the donor or the donor family to enhance the quality of life for other people.

Hence, bone graft compositions and methods are presently available and provide real benefits to patients in need thereof. Yet many advances may still be made to provide improved bone graft systems and methods for treating patients. The bone graft systems and treatment and manufacture methods described herein provide further solutions and answers to these outstanding needs.

### BRIEF SUMMARY

Bone is composed of organic and inorganic elements. By weight, bone is approximately 20% water. The weight of dry bone is made up of inorganic minerals such as calcium phosphate (e.g. about 65-70% of the weight) and an organic matrix of fibrous protein and collagen (e.g. about 30-35% of the weight). Both non-demineralized and demineralized bone can be used for grafting purposes.

Embodiments of the present invention encompass bone graft compositions containing mixtures of non-demineralized and demineralized bone, such that the compositions provide a bone allograft material having consistent calcium content, certain mechanical properties and handling characteristics, and desired biological activities. The bone graft compositions may include an amount of bone material, such as demineralized cortical bone, in the form of ribbon-shaped particles. The ribbon-shaped particles may intertwine or physically bond together. The interaction of ribbon-shaped particles with each other and with other particles may facilitate superior handling characteristics. The bone graft compositions may be easily handled and inserted into a container, such as a spine cage. The bone graft compositions may be easily compactable and moldable, and as a result, the bone graft compositions may be easily added to the container in the appropriate and desired amounts. The bone graft composition may exclude synthetic components, non-naturally-derived components, or components not derived from a donor.

In one aspect, embodiments of the present invention encompass composite bone graft materials, and methods for their use and manufacture. An exemplary method of manufacturing a composite bone graft material for administration to a treatment site of a human patient may include selecting a target calcium content or handling characteristic of the bone graft material, obtaining a first amount of demineralized cortical bone material from a donor. The demineralized cortical bone may include non-spherical particles. The method may also include obtaining a second amount of demineralized cancellous bone material from the donor. The method may further include obtaining a third amount of non-demineralized cortical bone material from the donor. The first amount, the second amount, and the third amount may be combined so as to obtain the bone graft composition having the target calcium content or handling characteristic. The demineralized cortical bone, the demineralized cancellous bone, and the non-demineralized cortical bone may all be obtained from the same cadaveric donor.

In these or other embodiments, the first amount of demineralized cortical bone material may be selected based on the target calcium content or handling characteristic. Relatedly, the third amount of non-demineralized cortical bone material may be selected based on the target calcium content or handling characteristic. The target calcium content may be between about 10% and about 15%. In some instances, a ratio of the first amount of demineralized cortical bone to the third amount of non-demineralized cortical bone may be selected based on the target calcium content or handling characteristic. In some instances, a ratio of the first amount of demineralized cortical bone to the second amount of demineralized cancellous bone may be selected based on a desired handling characteristic. In some embodiments, the donor is an allogeneic cadaveric donor. In some embodiments, the composite bone graft material includes tissue obtained from the patient.

The non-spherical, demineralized cortical bone material may include ribbon-shaped particles. Embodiments may include forming the ribbon-shaped particles from a non-demineralized cortical bone section, which may be used as a starting material and not in the final bone graft composition. The cortical bone section may have a length from about 20 mm to about 40 mm, a thickness from about 10 mm to about 30 mm, and a width from about 2 mm to about 4 mm.

The non-demineralized ribbons may have a minimum size of about 2 mm. A sieve with a pore size may be used to separate the non-demineralized ribbons from sawdust-like particles that are smaller than the pore size. The non-demineralized ribbons may be retained by the sieve, while smaller particles pass through the sieve. The non-demineralized ribbons may be demineralized to form the first amount of demineralized cortical bone material. The final composition may exclude non-demineralized ribbons.

Prior to the combining step, the method may include seeding the mesenchymal stem cells onto the demineralized bone material. The method may include seeding a stromal vascular fraction onto the demineralized bone material, and the stromal vascular fraction may include mesenchymal stem cells and unwanted cells. In these or other embodiments, the method may include incubating the mesenchymal stem cells on the demineralized bone material for a period of time to allow the mesenchymal stem cells to adhere to the demineralized bone material. The method may include rinsing the seeded demineralized bone material to remove the unwanted cells from the demineralized bone material. The demineralized bone may be either the cancellous bone or the cortical ribbons.

The first amount of demineralized cortical bone material may be about 50% of the volume of the bone graft composition. The second amount of demineralized cancellous bone material may include about 40% of the volume of the bone graft composition. The third amount of the non-demineralized cortical bone material may be between about 9% and about 11% of the volume of the bone graft composition. In some embodiments, the third amount may be about 10% by volume.

In addition, the first amount of demineralized cortical bone material, the second amount of demineralized cancellous bone material, and the third amount of non-demineralized cortical bone material may be any amount described herein. The sizes of the bone materials may be any sizes described herein. Indeed, the demineralized cortical bone material, demineralized cancellous bone material, and non-demineralized cortical bone material may be any combination of amounts and sizes described herein. Embodiments of the method may exclude obtaining non-demineralized cancellous bone material.

In another aspect, embodiments of the present invention may include a bone graft composition. The bone graft composition may include a first amount of demineralized cortical bone, a second amount of demineralized cancellous bone, and a third amount of non-demineralized cortical bone. The demineralized cortical bone, the demineralized cancellous bone, and the non-demineralized cortical bone may be obtained from the same cadaveric donor. Non-demineralized bone may be bone that has not contacted any acid and/or has not undergone either a complete or an incomplete demineralization process.

The demineralized cortical bone may include non-spherical particles. For example, the demineralized cortical bone may include ribbon-shaped particles. The demineralized cortical bone may be processed from non-demineralized cortical bone in the form of ribbon-shaped particles. The non-demineralized ribbon-shaped particles may have a minimum size of 2 mm. The minimum size, as used herein, describes the size of a hole in a sieve that does not allow a particle with the minimum size to pass through. The non-demineralized ribbon-shaped particle may have a minimum size of 2 mm, but the length, height, or thickness of the particle may be shorter or longer than 2 mm. The minimum size also depends on the configuration of the ribbon-shaped particle. The non-demineralized ribbon-shaped particle may be curled up rather than flat. After demineralizing the cortical bone, the demineralized ribbon-shaped particle may uncurl or be flat. The demineralized cortical bone may not include bone in powder form. In some embodiments, the bone graft composition may exclude demineralized cortical bone in powder form or may exclude greater than 5% by volume or greater than 10% by volume of demineralized cortical bone in powder form.

The first amount of demineralized cortical bone may be about 50% of the volume of the bone graft composition. The second amount of demineralized cancellous bone may be about 40% of the volume of the bone graft composition. The third amount of the non-demineralized cortical bone may be between about 9% and about 11% of the volume of the bone graft composition, including about 10%.

In these or other embodiments, the amount of demineralized cortical bone may have particle sizes selected based on needs of the patient, needs of the physician, or for other reasons. Small sizes may be easier for a physician to handle and may bind better with ribbon-shaped particles. For example, particles may have sizes between about 100 μm and 2 mm, between about 1 mm and about 2 mm, between about 120 μm and about 710 μm, between about 100 μm and 1 mm, between about 2 mm and about 3 mm, or between about 3 mm and about 4 mm. Smaller particles may increase biological activity. Cortical bone may contain growth factors, which may aid bone graft treatments. The volume of the amount of demineralized cortical bone may be based on targeted calcium content, growth factor content, or handling characteristics. For example, the amount may be between about 40% and about 60%, between about 30% and about 70%, between about 45% and about 55%, between about 49% and about 51%, or about 50% of the volume of the bone graft composition in embodiments. The calcium content of the demineralized cortical bone may be based on targeted calcium content or handling characteristics. For example, the demineralized cortical bone in the first amount may have a calcium content between about 0 wt. % and about 8 wt. %, between about 0 wt. % and about 4 wt. %, between about 4 wt. % and about 6 wt. %, between about 0 wt. % and about 2 wt. %, or about 0 wt. % in embodiments.

In some embodiments, the amount of demineralized cancellous bone may include particles having sizes based on needs of the patient, needs of the physician, or for other reasons. Large particles may be difficult for a physician to handle or to mix. Large particles may not bind well to ribbon-shaped particles. Examples of particle sizes may include between about 0.5 mm and about 2 mm, between about 0.1 mm and 0.5 mm, between about 0.5 mm and about 1 mm, between about 1 mm and about 1.5 mm, between about 1.5 mm and about 2 mm, between about 2 mm and about 4 mm, between about 4 mm and about 5 mm, between about 5 mm and 6 mm, or between about 6 mm and about 9 mm in embodiments. The second amount of demineralized cancellous bone may include mesenchymal stem cells seeded to the surface of the demineralized cancellous bone. The volume of the amount of demineralized cancellous bone may be chosen based on desired handling characteristics of the final product and/or the targeted calcium content. For example, the second amount may be between about 20% and about 60%, between about 30% and about 50%, between about 35% and about 45%, between about 38% and about 42%, or about 50% of the volume of the bone graft composition. The calcium content of the demineralized cancellous bone may be based on targeted calcium content or handling characteristics. For example, the calcium content may be between about 0% and about 8%, between about 0% and about 4%, between about 4% and about 6%, between about 0% and about 2%, or about 0% in embodiments. The demineralized cancellous bone may include bone matrix protein type 2 (BMP-2).

In some embodiments, the amount of non-demineralized cortical bone may have particles with sizes selected based on needs of the patient, needs of the physician, or for other reasons. Large particles may be difficult for a physician to handle or to mix. Examples of particle sizes may include between about 0.5 mm and about 1 mm, between about 0.1 mm and 0.5 mm, between about 0.5 mm and about 1 mm, between about 1 mm and about 1.5 mm, between about 1.5 mm and about 2 mm, between about 2 mm and about 4 mm, between about 4 mm and about 5 mm, between about 5 mm and 6 mm, or between about 6 mm and about 9 mm in embodiments. The volume of the amount of non-demineralized cortical bone may be chosen based on desired handling characteristics of the final product and/or the targeted calcium content. For example, the amount may be between about 5% and about 15%, between about 15% and about 30%, between about 7% and about 13%, between about 1% and about 5%, between about 9% and about 11%, or about 10% of the volume of the bone graft composition. The non-demineralized cortical bone in the first amount may have a calcium content selected based on handling characteristics or targeted calcium content. For example, the calcium content may be between about 20 wt. % and about 25 wt. %.

In these or other embodiments, the bone graft composition may have a calcium content between about 10 wt. % and about 19 wt. %, between about 10 wt. % and about 15 wt. %, between about 12 wt. % and about 17 wt. %, or about 15 wt. %. The calcium content of the bone graft composition may be measured by a residual calcium test or other known methods. The demineralized bone material may make up between about 25% and about 95%, between about 50% and about 75%, between about 75% and about 95%, or about 90% of the cortical bone in the bone graft composition in embodiments. The remainder of the cortical bone material may be non-demineralized bone material. In embodiments, the bone graft composition may not include non-demineralized cancellous bone. In some embodiments, the bone graft composition may not include greater than 5% by volume or greater than 10% by volume of non-demineralized cancellous bone.

In another aspect, embodiments of the present invention may include a method of treating a bone defect or other ailment in a patient. The method may include administering to the patient a bone graft composition that may include a first amount of demineralized cortical bone, a second amount of demineralized cancellous bone, and a third amount of non-demineralized cortical bone. The demineralized cortical bone, the demineralized cancellous bone, and the non-demineralized cortical bone may be obtained from the same cadaveric donor. The demineralized cortical bone may include ribbon-shaped particles. The bone graft composition may be administered to treat spinal problems. With some spinal problems, the spine may need to be fused. In these or other embodiments, the bone graft composition may be placed in a container, such as a spine cage. The spine cage may be applied to the patient, which may include placing the cage between vertebrae. Additionally, the bone graft composition may be used to treat nonunions or critical size defects. In these or other embodiments, the bone graft composition may be applied or administered to the bone defect or surrounding bone.

In yet another aspect, embodiments of the present invention may include a kit. The kit may include any bone graft composition described herein. The kit may also include a strainer, a vial with a lid, a tray, a tray lid, a box, instructions for use, information about the donor and/or recipient, a feedback form for a medical professional, or labels, or any combination thereof.

The above described and many other features and attendant advantages of embodiments of the present invention will become apparent and further understood by reference to the following detailed description when considered in conjunction with the accompanying drawings.

### BRIEF DESCRIPTION OF THE DRAWINGS

FIG. 1 depicts aspects of bone graft systems and methods according to embodiments of the present invention.

FIG. 2 shows the steps in a method of manufacturing a composite bone graft material according to embodiments of the present invention.

FIG. 3 shows operations in preparing demineralized cortical bone ribbons from non-demineralized cortical bone in embodiments.

FIG. 4 shows the steps in a method of treating a patient according to embodiments of the present invention.

FIG. 5 depicts aspects of bone graft systems and methods according to embodiments of the present invention.

FIGS. 6A, 6B, 6C, 6D, and 6E show images of bone material according to embodiments of the present invention.

### DETAILED DESCRIPTION

Embodiments of the present invention encompass bone graft compositions and methods for their use and manufacture. Bone graft compositions as disclosed herein are provided with selected calcium content and/or handling characteristics. An exemplary manufacturing method may include selecting a target calcium content or handling characteristic of a bone graft composition, selecting a first amount of demineralized bone material, selecting a second amount of demineralized bone material, selecting a third amount of non-demineralized bone material, and combining the first, second, and third amounts of bone material so as to obtain a bone graft composition having the target calcium content or handling characteristic.

Demineralization

Mineralized bone may be demineralized. For example the osteoid, which can be about 50% of the bone volume, is composed mainly of collagen. The mineralization of osteoid by inorganic mineral salts provides bone with its strength and rigidity. Bone contains several inorganic mineral components, such as calcium phosphate, calcium carbonate, magnesium, fluoride, sodium, and the like. Typical demineralization procedures involve removing such mineral components from bone. Any of a variety of techniques can be used to demineralize bone, including hydrochloric acid treatments, and the like. Demineralized bone matrix (DBM) refers to allograft bone that has had the majority of the inorganic mineral removed, leaving behind the organic collagen matrix. The American Association of Tissue Banks typically defines demineralized bone matrix as containing no more than 8 wt. % residual calcium as determined by standard methods. In this sense, a fully demineralized bone tissue can be considered to have no more than 8 wt. % residual calcium. In some embodiments, the residual calcium may be less than 4 wt. %, 2 wt. %, or 1 wt. %. The process of demineralizing may be from 60 to 75 minutes.

After demineralization, bone matrix protein type 2 (BMP-2) may still be present in cancellous or cortical bone. In demineralized cancellous bone, BMP-2 may be present at about 9 ng/g to 13 ng/g.

Cortical Bone

Cortical bone, also known as compact bone, can be found in the outer shell portion of various bones. Cortical bone is typically, dense, hard, strong, and stiff. Cortical bone may include bone growth factors.

Cancellous Bone

Cancellous bone, also known as spongy bone, can be found at the end of long bones.

Cancellous bone is typically less dense, softer, weaker, and less stiff than cortical bone.

Mineral Content of Bone

Cortical bone and cancellous bone can be harvested from a donor individual using standard techniques. The mineral or calcium content of the harvested bone may vary. In some cases, cortical bone is about 95% mineralized and cancellous bone is about 35-45% mineralized.

In some cases, cortical bone is about 73.2 wt. % mineral content, and cancellous bone is about 71.5 wt. % mineral content. In some cases, the mineral content of the starting bone material is about 25 wt. %, prior to demineralization.

Composite Bone Materials

Embodiments of the present invention encompass bone materials containing various mixtures of mineralized (or non-demineralized) bone combined with demineralized bone. For example, bone compositions may include fully demineralized bone (e.g. cortical and/or cancellous) combined with non-demineralized bone (e.g. cortical and/or cancellous). Non-demineralized bone may be bone that has not undergone any demineralization, including treatment with acid. Demineralized and non-demineralized bone can be combined at certain ratios to provide bone allograft material having consistent calcium content and/or preferable handling characteristics.

Compositions and methods may exclude non-demineralized cancellous bone. Excluding non-demineralized cancellous bone may allow for easier targeting of the final calcium content. In addition, cancellous bone is generally spongy and may increase the volume of the composition, making the composition harder to be well mixed with other more granular components. Furthermore, adding in non-demineralized cancellous bone may interfere with the ability of the demineralized cortical bone ribbons to intertwine with each other and the other components.

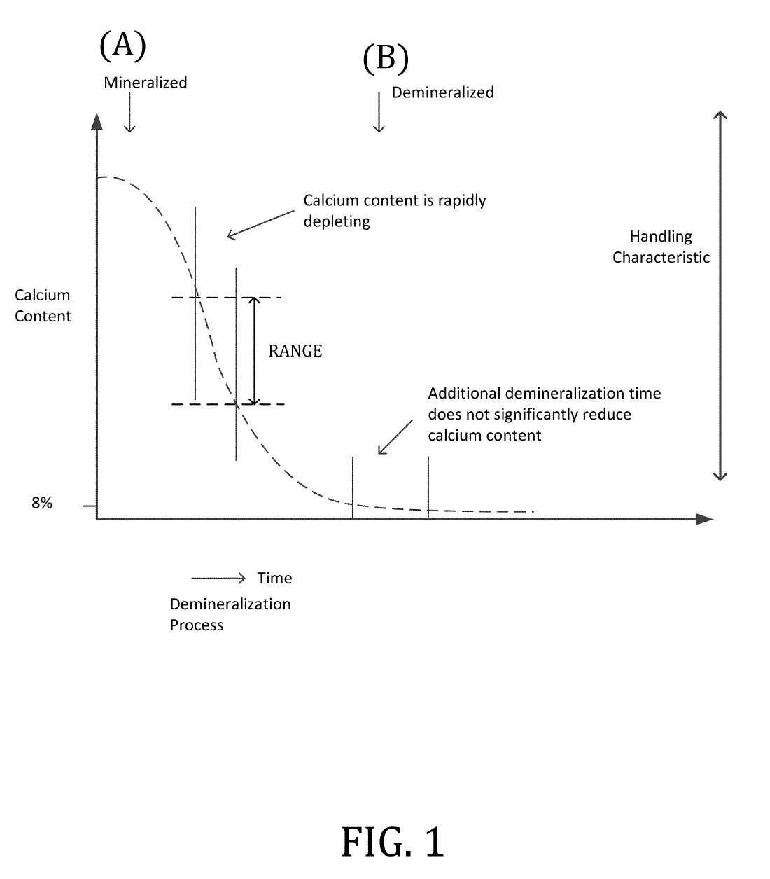

Turning now to the drawings, FIG. 1 depicts aspects of bone composite systems and methods according to embodiments of the present invention. As shown here, as a typical demineralization method proceeds, the amount of calcium in the bone is rapidly depleted. What is more, the acid concentration used, the duration of the demineralization process, and the process temperature are factors which can operate to impact the residual calcium content in the bone. Moreover, there may be variation in the bone density (e.g. due to donor age and/or bone location) as well as in the bone particle size. Hence, it may be difficult to accurately obtain a partially demineralized bone material having a calcium content which is within the specified range, or that is at a particular desired or selected value within the range.

Exemplary bone allograft compositions as disclosed herein contain mineralized bone (A) and additionally demineralized bone (B). Hence, the bone allograft composition can have a consistent calcium content. Typically, the demineralized bone is provided as a demineralized bone matrix, or allograft bone which has had inorganic mineral removed, leaving behind the organic collagen matrix. As a result of the demineralization process, the DBM is more biologically active (e.g. BMPs, including BMP-2, were activated during demineralization process) than non-demineralized bone grafts. Conversely the mechanical or structural integrity properties of demineralized bone may be significantly diminished as compared to mineralized bone.

Typically, cortical bone and cancellous bone are separated from one another, and then demineralized. For example, the cortical bone and cancellous bone can be demineralized in separate batches.

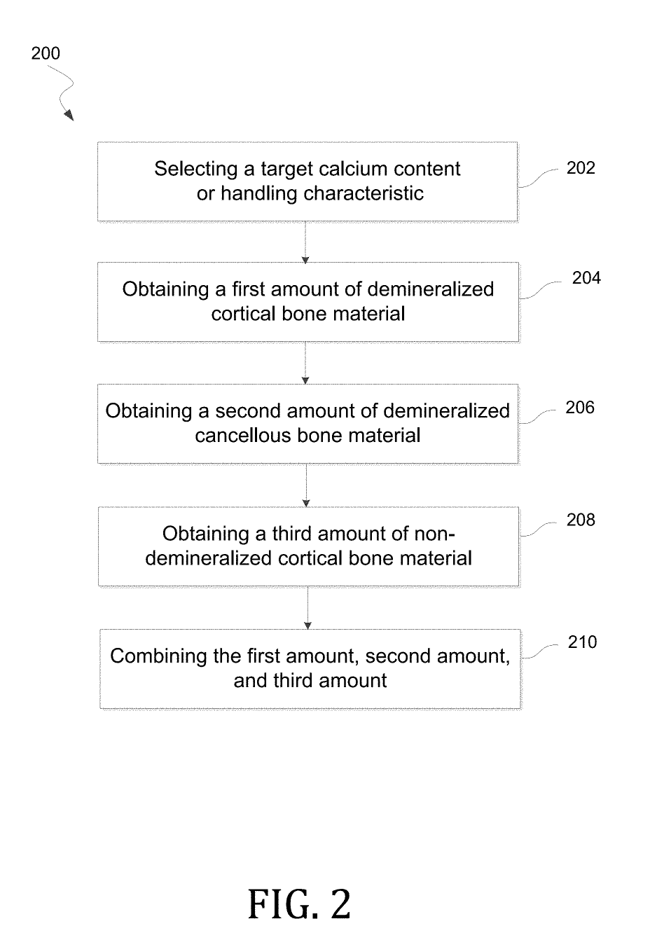

FIG. 2 shows the operations in a method 200 according to embodiments of the present invention.

At block 202, method 200 may include selecting a target calcium content or handling characteristic. The target calcium content may be between about 10% and about 15%. The handling characteristic may be a composition that sticks together well and is not too spongy.

At block 204, method 200 may include selecting a first amount of demineralized cortical bone material. In these or other embodiments, the bone material and any bone material described herein may be from a donor. The demineralized cortical bone material may be in the form of ribbons instead of spherical particles. The demineralized cortical bone material may be ribbons formed from non-demineralized cortical bone ribbons that do not fit through a sieve with a certain diameter hole. For example, the size of the hole in the sieve may be 1 mm, 2 mm, 3 mm, 4 mm, or 5 mm in embodiments. The ribbon may be curled up so that the length of an uncurled ribbon may be greater than the size of the hole in the sieve. The size of the hole in the sieve describes the size of the ribbon rather than a specific length, width, or thickness of the uncurled ribbon.

A ribbon may be a narrow, long, and thin piece characterized by a width, a length, and a thickness. The length may be over 1 time, from 1 time to 3 times, from 3 times to 6 times, from 6 times to 10 times, from 10 times to 25 times, from 25 times to 100 times, from 100 times to 600 times, over 600 times the width, or any combination of ranges in embodiments. The length may be over 1 time, from 1 time to 2.5 times, from 2.5 times to 10 times, from 10 times to 15 times, from 15 times to 100 times, from 100 times to 500 times, from 500 times to 600 times, over 600 times the thickness, or any combination of ranges in embodiments. The thickness of the non-demineralized ribbon may be from 0.05 mm to 5 mm, from 0.05 mm to 0.1 mm, from 0.1 mm to 0.5 mm, from 0.5 mm to 1 mm, from 1 mm to 2 mm, from 2 mm to 4 mm, from 4 mm to 5 mm, or any combination of ranges in embodiments. The length of the non-demineralized ribbon may be from 5 mm to 60 mm, from 5 mm to 10 mm, from 10 mm to 20 mm, from 20 mm to 30 mm, from 30 mm to 40 mm, from 40 mm to 50 mm, from 50 mm to 60 mm, more than 60 mm, or any combination of the ranges in embodiments. The width of the non-demineralize d ribbon may be from 0.1 mm to 8 mm, from 0.1 mm to 2 mm, from 2 mm to 5 mm, from 5 mm to 8 mm, from 8 mm to 10 mm, more than 10 mm, or any combination of ranges in embodiments.

The ribbons of non-demineralized cortical bone material may be prepared from a cortical block segment. The block segment may have a length from 5 mm to 60 mm, from 5 mm to 10 mm, from 10 mm to 20 mm, from 20 mm to 30 mm, from 30 mm to 40 mm, from 40 mm to 50 mm, from 50 mm to 60 mm, more than 60 mm, or any combination of the ranges in embodiments. The block segment may have a width from 0.1 mm to 8 mm, from 0.1 mm to 2 mm, from 2 mm to 5 mm, from 5 mm to 8 mm, from 8 mm to 10 mm, more than 10 mm, about 3 mm, or any combination of ranges in embodiments. The thickness of the block segment may be from 1 mm to 10 mm, from 10 mm to 20 mm, from 20 mm to 30 mm, from 30 mm to 40 mm, greater than 40 mm, or any combination of ranges in embodiments. The block segment may be shaved with a knife edge, shaver, or suitable tool to produce a ribbon of cortical bone material.

The length and width of the resulting ribbon may be equal or about equal to the length and width of the original block segment before shaving. The thickness of the resulting ribbon may be less than the thickness of the block segment. The thickness of the resulting ribbon may be from 0.05 mm to 5 mm, from 0.05 mm to 0.1 mm, from 0.1 mm to 0.5 mm, from 0.5 mm to 1 mm, from 1 mm to 2 mm, from 2 mm to 4 mm, from 4 mm to 5 mm, or any combination of ranges in embodiments. The ribbons may be curled after shaving. The ribbons may then be demineralized and may uncurl as a result of demineralization.

The non-demineralized cortical bone ribbons may be demineralized by any technique described herein or any suitable technique. The first amount of the demineralized cortical bone may be measured by using a scoop of known volume or any other suitable vessel of known volume. Additional details for preparing the demineralized cortical bone ribbons are discussed below.

At block 206, method 200 may include selecting a second amount of demineralized cancellous bone material. The demineralized cancellous bone material may be spherical particles or nearly spherical particles. Spherical particles or nearly spherical particles may be particles that appear spherical to the naked eye. The particles may have a distance from the center of mass of the particle to the surface of the particle that does not vary by more than 5% or more than 10%. The particles may have a size from 0.5 mm to 2 mm, from 0.5 mm to 1 mm, from 1 mm to 2 mm, from 2 mm to 3 mm, or from 3 mm to 4 mm in embodiments. The particle sizes may not be the diameter of the particles, because some particles may not be spherical. The size ranges may refer to the sizes of holes in sieves used to separate particles rather than a measure of a specific dimension of the particles. The minimum size of a range may describe the size of pores in a sieve that retains the particles, while the maximum size of the range may describe the size of pores in another sieve that allows the particles to pass through. The second amount may be measured by using a scoop of known volume or any suitable vessel of known volume.

At block 208, method 200 may include obtaining a third amount of non-demineralized cortical bone. The non-demineralized cortical bone may include ground cortical bone. The non-demineralized cortical bone may include powder or particles with a characteristic size from 0.5 mm to 1 mm, from 1.0 mm to 1.5 mm, or from 1.5 mm to 2.0 mm. The non-demineralized cortical bone may be radiopaque and may provide contrast for x-rays. The third amount may be measured by using a scoop of known volume or any suitable vessel of known volume.

At block 210, method 200 may include combining the first amount, the second amount, and the third amount. By combining non-demineralized and demineralized bone material, it is possible to obtain a resulting mixture having desirable handling characteristics, calcium content, and BMP activity. In some embodiments, all three amounts are combined at the same time or nearly the same time. In other embodiments, the demineralized cancellous bone and the non-demineralized cortical bone may first be mixed before combining with the non-demineralized cortical bone ribbons. In some cases, the non-demineralized cortical bone and the demineralized cortical bone ribbons are combined before adding in the demineralized cancellous bone. In other embodiments, the demineralized cortical bone ribbons and the demineralized cancellous bone are combined before mixing in the non-demineralized cortical bone. The amounts may be agitated to partially, mostly, or completely homogenize the composition.

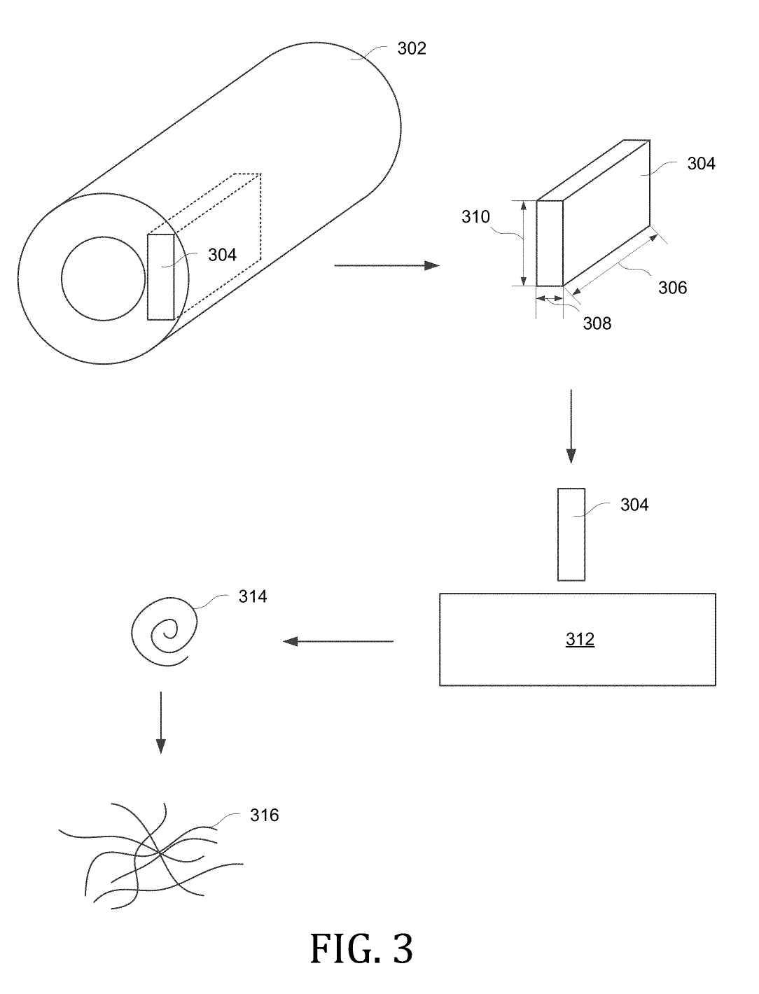

FIG. 3 shows the preparation of demineralized cortical bone ribbons from a cortical bone 302. A block segment 304 is cut from cortical bone 302. Block segment 304 has a length 306, a width 308, and a thickness 310. Block segment 304 may be taken to a shaver 312, which forms ribbons of cortical bone with lengths and widths similar to that of block segment 304. Shaver 312 forms cortical bone pieces with similar thicknesses. The ribbons may be similar to ribbon 314, which is curled up after being formed. Ribbon 314 may then be demineralized to form demineralized cortical bone ribbon 316. Demineralized cortical bone ribbon 316 may uncurl after demineralization. To prepare a sufficient number of demineralized cortical bone ribbons, several block segments, including hundreds of block segments, may be obtained from cortical bone, which may be from different bones of a donor. Different block segments may have different dimensions.

In some embodiments, particle sizes and/or volume percentages of components of the bone graft composition may be selected based on handling characteristics of the bone graft composition. Such handling characteristics may include compressibility and cohesion characteristics. Compressibility characteristics for bone graft compositions may include whether the composition is more like sand or more like a sponge. Cohesion characteristics for bone graft compositions may include compositions whether the composition sticks together or does not stick together. Compositions that are more like sand and do not stick together may be difficult for a physician to handle and administer to only the treatment site. Product that is too spongy may be harder for physicians to apply in consistent amounts across different treatments in part because in some instances, a physician may compress the spongy product more than in other instances. The resulting bone graft compositions may have putty-like properties. The compositions may stick together, while being moldable. In embodiments, the compositions may fit into a spinal cage. The composition in the spinal cage may not fall out when the spinal cage is agitated, improving handling of the composition of spinal cage by the physician.

In some cases, composite materials may include components which are present in an amount that is within a volume percentage range. For example, demineralized cancellous bone may be present within a range between about 30% and about 70%, between about 40% and about 60%, between about 45% and about 55%, between about 48% and about 52%, or about 50%. The non-demineralized cancellous bone may be present within a range between about 30% and about 70%, between about 40% and about 60%, between about 45% and about 55%, between about 48% and about 52%, or about 50%. The demineralized cortical bone may be present within a range between about 10% and about 40%, between about 10% and about 30%, between about 15% and about 25%, between about 19% and about 21%, or about 20%. Such volumes may be based on the volume of the bone graft composition product.

In an exemplary embodiment, a composite bone material may have a calcium content within a range from about 10 wt. % to about 15 wt. %. In these or other embodiments, the bone graft composition may have a calcium content between about 10 wt. % and about 19 wt. %, between about 12 wt. % and about 17 wt. %, or about 15 wt. %. In some cases, an amount of mineralized cancellous bone present in the composite bone material may have a calcium content of about 20 wt. %. In some cases, an amount of demineralized cancellous bone present in the composite bone material may have a calcium content within a range from about 0 wt. % (or undetected) to about 8 wt. %. In some cases, an amount of mineralized cortical bone present in the composite bone material may have a calcium content of about 25 wt. %. In some cases, an amount of cortical demineralized bone present in the composite bone material may have a calcium content within a range from about 0 wt. % (or undetected) to about 8 wt. %.

Examples of particle sizes of demineralized cancellous bone material may include between about 0.1 mm and about 9 mm, between about 2 mm and about 8 mm, between about 1 mm and about 7 mm, between about 1 mm and about 6 mm, between about 1 mm and about 5 mm, between about 0.1 mm and about 4 mm, between about 1 mm and about 4 mm, or between about 0.1 mm and about 1 mm, or between about 0.5 mm and about 4 mm in embodiments. Examples of particles sizes of cortical bone may include between about 100 μm and 2 mm, between about 1 mm and about 2 mm, between about 120 μm and about 710 μm, or between about 100 μm and 1 mm. Smaller particle sizes may result in more BMPs being activated. Particle sizes may be obtained using a series of sieves to remove particles smaller and larger than a desired range.

According to some embodiments, instead of adding a patient's own cancellous bone material to an implant graft composition to treat a fracture or other bone defect during a surgical procedure, it is possible to use non-demineralized cancellous bone in a composite bone material as discussed elsewhere herein.

In some cases, the composite bone material will include bone obtained from an allogeneic donor. In some cases, both the demineralized and the mineralized components can be harvested from a common donor and combined to provide the composite bone material.

Relatedly, the composite bone material can include cells (e.g. adult mesenchymal stem cells) obtained from the same donor. It has been observed that cells such as mesenchymal stem cells may exhibit an affinity for adhering with demineralized bone. The mesenchymal stem cells may adhere to demineralized cancellous bone or the demineralized cortical ribbons. In some cases, the composite bone material may include bone obtained from a recipient patient. Hence, a composite bone material may include autologous demineralized and/or mineralized bone.

In some embodiments, methods of manufacturing composite bone graft material may include seeding demineralized bone material with a stromal vascular fraction. The stromal vascular fraction may be formed by digesting adipose tissue. Digesting the adipose tissue may include making a collagenase I solution and filtering the solution, and mixing the adipose solution with the collagenase solution. The adipose solution with the collagenase I solution may be agitated in a shaker flask. This may provide the adipose tissue with a visually smooth appearance. The method may include aspirating a supernatant containing mature adipocytes so as to provide a pellet that is the stromal vascular fraction.

The stromal vascular fraction may include mesenchymal stem cells and other cells, which may be unwanted or unneeded in embodiments of the invention. Unwanted cells may include hematopoietic stem cells and other stromal cells. In these or other embodiments, methods may include incubating the mesenchymal stem cells on the demineralized bone material for a period of time to allow the mesenchymal stem cells to adhere to the demineralized bone material. Methods may include rinsing the seeded demineralized bone material to remove all, substantially all, or a portion of the unwanted cells from the demineralized bone material in embodiments. Methods involving mesenchymal stem cells may be as disclosed in U.S. Patent Application Ser. Nos. 61/116,484, 61/285,463, Ser. No. 12/612,583, Ser. No. 14/880,563, Ser. No. 14/880,675, Ser. No. 12/965,335, Ser. No. 14/207,220, Ser. No. 14/938,173, Ser. No. 14/923,087, Ser. No. 14/081,913, Ser. No. 14/877,392, Ser. No. 14/187,093, Ser. No. 14/875,258, Ser. No. 14/210,111, Ser. No. 14/858,386, Ser. No. 14/940,798, Ser. No. 14/938,173, and Ser. No. 15/235,607, the entire contents of all are incorporated herein by reference for all purposes.

In some cases, the bone graft composition may be administered to a patient as a flowable, syringeable, putty-like material. For example, a putty-like moldable matrix can be delivered through a cannula or other syringe attachment to a treatment site. Such bone matrix compositions may be used as how soft tissue matrix compositions are used and formed in U.S. patent application Ser. No. 13/712,295 filed Dec. 12, 2012 (now U.S. Pat. No. 9,162,011), the entire content of which is incorporated herein by reference for all purposes. In some cases, the bone material and/or mesenchymal stem cells may be present in a morselized form. Hence, compositions and methods as disclosed herein may include a soft tissue or skin matrix material combined with stem cell morsels, so as to form a bone putty. A putty formulation may have good handling characteristics. For example, such morsels or putty compositions may stay in place upon implantation. Relatedly, such morsels or putty compositions may persist at the site of the application (e.g., bone defect area) and resist removal by irrigation and/or contact with blood. In some instances, flowable decellularized skin or de-epidermalized skin (or other soft tissue) can provide and effective carrier to hold demineralized bone material and/or mesenchymal stem cells in place and prevent their migration.

Embodiments of the present invention may encompass delivering the bone graft composition combined with a carrier to a treatment site of the patient. In some cases, the carrier is derived from a human donor and includes an organic phase of a decellularized adipose tissue that has been exposed to alkaline organic solution. Such methods and compositions may be similar to those taught in U.S. patent application Ser. No. 13/970,324 filed Aug. 19, 2013, the entire content of which is incorporated herein by reference for all purposes. In these or other embodiments, methods may include administering treatment material combined with a matrix to a treatment site of a patient. The matrix may include a processed organic phase of decellularized adipose tissue that is substantially free of a stromal vascular fraction. In some cases, the adipose component includes an adipose derived carrier.

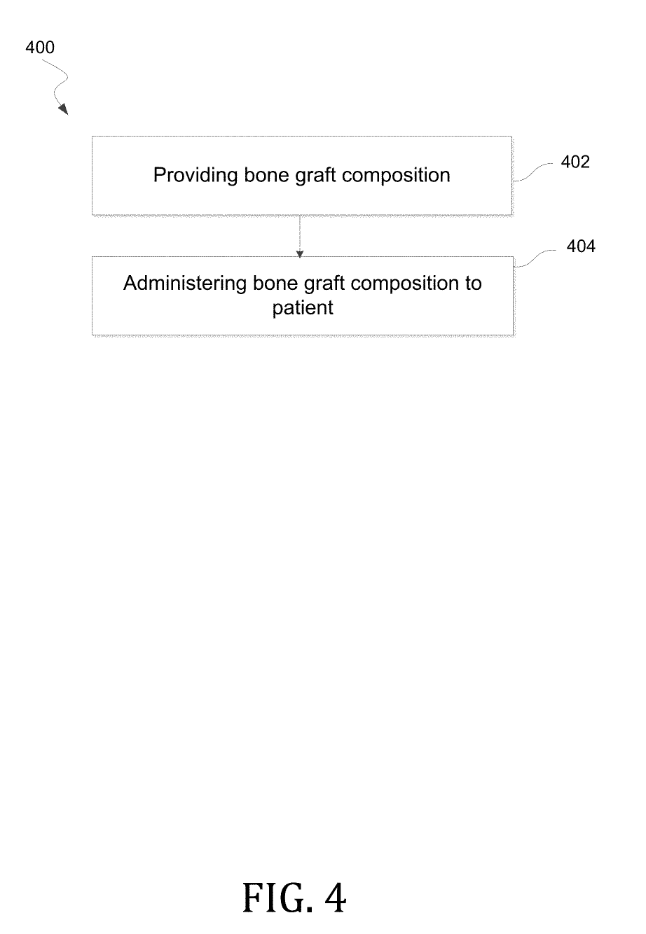

FIG. 4 shows a method 400 of treating a bone defect in a patient according to embodiments. The method 400 may include providing a bone graft composition 402. In these or other embodiments, the bone graft composition may include any of the bone graft compositions described herein. The method 400 may include administering the bone graft composition to the patient 404. Administering the bone graft composition may include applying the bone graft composition to the patient. The bone graft composition may be molded to the shape of the administration site before or after the bone graft composition is applied.

### EXAMPLE 1

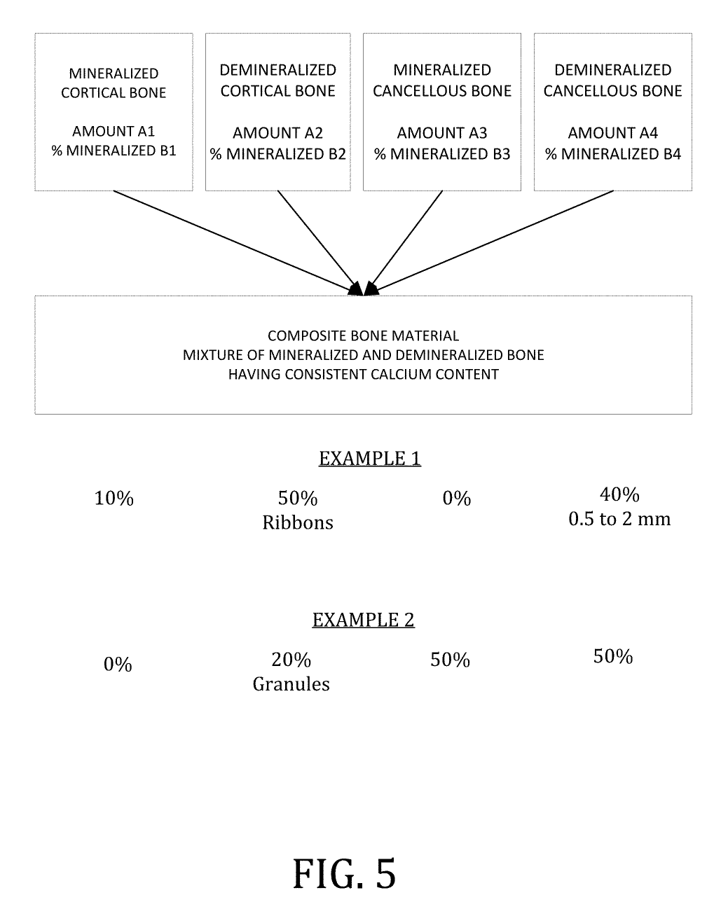

In one example (FIG. 5), a composite bone material included 50% demineralized cortical bone (e.g. ribbons from non-demineralized ribbons greater than 2 mm in size), 40% fully demineralized cancellous bone (e.g. 0.5 mm to 2 mm particle size), and 10% non-demineralized cortical bone (e.g. 0.5 mm to 1 mm particle size). Particle size ranges for the demineralized cancellous bone and non-demineralized cortical bone were obtained by using two sieves to separate out ground bone material. The demineralized cortical bone was present as ribbon-shaped particles before combining with the demineralized cancellous bone and the mineralized cortical bone. The ribbon-shaped particle sizes were obtained using one sieve on non-demineralized ribbon shaped-particles and then subsequently demineralizing. The ribbon-shaped particles intertwined with other ribbon-shaped particles.

### EXAMPLE 2

In another example (FIG. 5), a composite bone material included amounts of bone material as granules or powder, without any ribbons. The composite include 20% demineralized cortical bone, 50% non-demineralized cancellous bone, and 50% demineralized cancellous bone. The volume percentages total more than 100% because the non-demineralized cancellous bone contains voids, which may house other bone material in the final composition.

### EXAMPLE 3

The handling characteristics of the allograft material in Examples 1 and 2 were evaluated. With the material in Example 1, it was observed that the allograft material exhibited consistent and satisfying handling characteristics. In one instance, the allograft material was fit into a cage similar to a spinal cage. The cage with the allograft material was agitated. The allograft material stayed in the cage. By contrast, the allograft material of Example 2 with granular particles instead of ribbons had material fall out of the cage when agitated. Hence, by combining demineralized cortical bone in ribbon-shaped particles, fully demineralized bone material (e.g. DBM) with a non-demineralized bone matrix material, it is possible to create a very consistent partially demineralized bone product that may have improved handling characteristics. The intertwining of ribbons may help stabilize the material inside the cage. Relatedly, the product can have a consistent calcium content.

### EXAMPLE 4

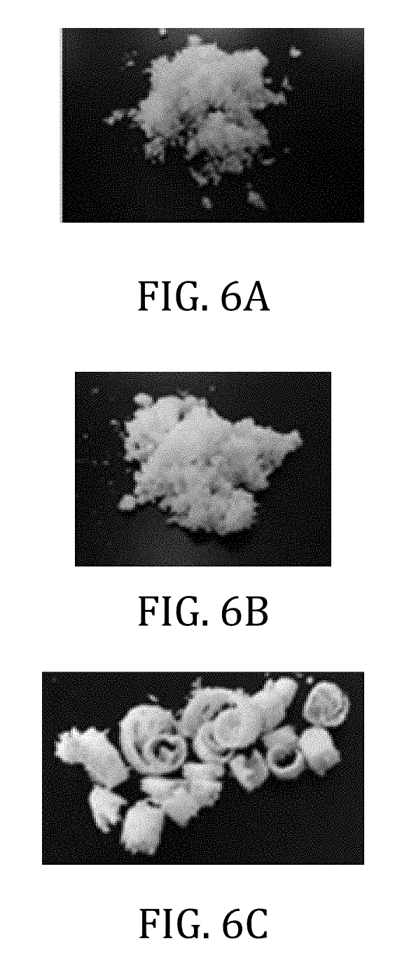

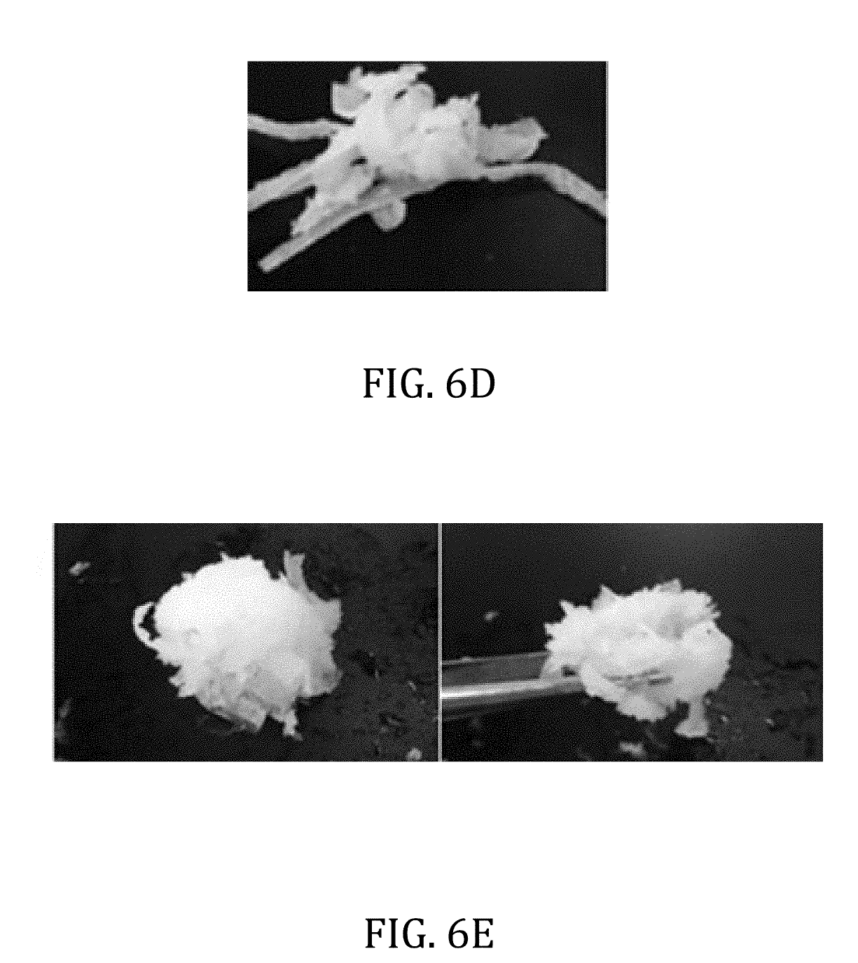

Images of different bone materials were taken. FIG. 6A shows demineralized cancellous bone. The demineralized cancellous bone has a size of between 0.5 mm and 2 mm. FIG. 6B shows non-demineralized cortical bone. The non-demineralized cortical bone has a size between 0.5 and 1 mm. The non-demineralized cortical bone is mostly spherical. FIG. 6C shows non-demineralized cortical bone as ribbons. The non-demineralized cortical bone ribbons have a size of greater than 2 mm. In other words, the non-demineralized cortical bone ribbons cannot pass through a sieve with a 2 mm diameter opening. FIG. 6D shows the cortical bone after demineralization. After demineralization, some of the ribbon-shaped particles uncurl. The demineralized particles also become more translucent. FIG. 6E shows a composition of the demineralized cortical bone ribbons along with demineralized cancellous bone seeded with stem cells, and non-demineralized cortical bone. The composition shows a mass of the particles. The ribbon-shaped particles are intertwined with each other with the cancellous bone and the non-demineralized cortical bone interspersed throughout the mass.

### EXAMPLE 5

A composition of 50% demineralized cortical bone ribbons, 40% demineralized cancellous bone, and 10% non-demineralized cortical bone was prepared by methods described herein. The compositions were provided to 24 individuals, including surgeons and distributors. The individuals provided comments regarding their impressions of the composition, often in comparison to current market offerings. These comments were scored on a three-point scale. Comments that expressed negative views of the composition were awarded one point. Comments that expressed neutral views of the composition were awarded two points. Comments that expressed positive views were awarded three points. Using this scoring scale, the average score was 2.7. Nineteen of the respondents provided positive feedback, with three respondents having neutral feedback and two respondents having negative feedback. Positive feedback included “fantastic handling characteristics” and “loved handling characteristics” from two surgeons. This example showed that the composition of demineralized cortical bone ribbons, demineralized cancellous bone particles, and non-demineralized cortical bone particles have superior handling and other characteristics.

### EXAMPLE 6

Different volume percentages of demineralized cortical bone, non-demineralized cortical bone, and demineralized cancellous bone in a bone graft composition are tested for different handling characteristics.

When referring to the calcium content in the product, there may be various types of calcium, such as calcium phosphate, calcium carbonate, and the like. For example, some bone material is formed mostly of calcium phosphate in the chemical arrangement termed calcium hydroxylapatite.

All patents, patent publications, patent applications, journal articles, books, technical references, and the like discussed in the instant disclosure are incorporated herein by reference in their entirety for all purposes.

It is to be understood that the figures and descriptions of the invention have been simplified to illustrate elements that are relevant for a clear understanding of the invention. It should be appreciated that the figures are presented for illustrative purposes and not as construction drawings. Omitted details and modifications or alternative embodiments are within the purview of persons of ordinary skill in the art.

It can be appreciated that, in certain aspects of the invention, a single component may be replaced by multiple components, and multiple components may be replaced by a single component, to provide an element or structure or to perform a given function or functions. Except where such substitution would not be operative to practice certain embodiments of the invention, such substitution is considered within the scope of the invention.

The examples presented herein are intended to illustrate potential and specific implementations of the invention. It can be appreciated that the examples are intended primarily for purposes of illustration of the invention for those skilled in the art. There may be variations to these diagrams or the operations described herein without departing from the spirit of the invention. For instance, in certain cases, method steps or operations may be performed or executed in differing order, or operations may be added, deleted or modified.

Different arrangements of the components depicted in the drawings or described above, as well as components and steps not shown or described are possible. Similarly, some features and sub-combinations are useful and may be employed without reference to other features and sub-combinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications can be made without departing from the scope of the claims below.

Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included.

1. A bone graft composition, comprising:
a first amount of demineralized cortical bone;
a second amount of demineralized cancellous bone; and
a third amount of non-demineralized cortical bone, wherein:
the demineralized cortical bone, the demineralized cancellous bone, and the non-demineralized cortical bone are obtained from the same cadaveric donor,
the demineralized cortical bone comprises non-spherical particles,
the bone graft composition has a calcium content between about 10 wt. % and about 15 wt. %, and
the bone graft composition excludes at least one of non-naturally derived components or components not derived from the cadaveric donor, wherein:
the demineralized cortical bone comprises ribbon-shaped particles, and
the bone graft composition excludes ribbon-shaped particles comprising non-demineralized cortical bone.

2. (canceled)

3. The bone graft composition according to claim 1, wherein the ribbon-shaped particles have a minimum size of 2 mm before the demineralized cortical bone is demineralized.

4. The bone graft composition according to claim 1, wherein the demineralized cancellous bone comprises spherical particles.

5. The bone graft composition according to claim 1, wherein the non-demineralized cortical bone comprises powder.

6. The bone graft composition according to claim 1, wherein the demineralized cortical bone does not comprise powder.

7. The bone graft composition according to claim 1, wherein the first amount of demineralized cortical bone comprises mesenchymal stem cells seeded to a surface of the demineralized cortical bone.

8. The bone graft composition according to claim 1, wherein the second amount of demineralized cancellous bone comprises mesenchymal stem cells seeded to a surface of the demineralized cancellous bone.

9. (canceled)

10. The bone graft composition according to claim 1, wherein:
the demineralized cortical bone comprises ribbon-shaped particles formed from non-demineralized cortical bone ribbons having a minimum size of about 2 mm before being demineralized,
the demineralized cancellous bone comprises particles having sizes between about 0.5 mm and about 2 mm, and
the non-demineralized cortical bone comprises particles having sizes between about 0.5 mm and about 1 mm.

11. The bone graft composition according to claim 1, wherein:
the first amount is about 50% of the volume of the bone graft composition, and
the second amount is about 40% of the volume of the bone graft composition.

12. The bone graft composition according to claim 1, wherein the third amount is between about 9% and about 11% of the volume of the bone graft composition.

13. The bone graft composition according to claim 1, wherein:
the first amount is about 50% of the volume of the bone graft composition,
the second amount is about 40% of the volume of the bone graft composition, and
the third amount is about 10% of the volume of the bone graft composition.

14. The bone graft composition according to claim 1, wherein the demineralized cancellous bone has a residual calcium amount of less than or equal to 8 wt. %.

15. The bone graft composition according to claim 1, wherein the demineralized cortical bone has a residual calcium amount of less than or equal to 8 wt. %.

16. The bone graft composition according to claim 1, wherein the demineralized cancellous bone comprises bone morphogenic protein type 2 (BMP-2).

17. The bone graft composition according to claim 1, wherein:
the bone graft composition does not comprise non-demineralized cancellous bone, and
the bone graft composition sticks together, is moldable, and is compactable.

18.-40. (canceled)

41. The bone graft composition according to claim 1, wherein the bone graft composition excludes non-naturally derived components.

42. The bone graft composition according to claim 1, wherein the bone graft composition excludes components not derived from the cadaveric donor.

43. The bone graft composition according to claim 1, wherein the bone graft composition is a flowable, syringeable, and putty-like material.

44. A kit comprising the bone graft composition according to claim 1, and at least one of a strainer, a vial with a lid, a tray, a tray lid, a box, instructions for use, information about the donor and/or recipient, a feedback form for a medical professional, or a label.

45. The bone graft composition according to claim 1, wherein the bone graft composition is a flowable, syringeable, putty-like material.
